(19) United States
(12) Patent Application Publication
(10) Pub. No.: US 2005/0131355 A1
(43) Pub. Date: Jun. 16, 2005

(54) Injection or infusion device with refined surface

(76) Inventors: Fritz Kirchhofer, Sumiswald (CH); Beat Steffen, Saanen (CH)

Correspondence Address: David E. Bruhn, Esq.;DORSEY & WHITNEY LLP, Intellectual Property Department, 50 South Sixth Street, Suite 1500, Minneapolis, MN, 55402-1498, US

(21) Appl. No.: 11/010,192

(22) Filed: Dec. 10, 2004

Related U.S. Application Data
(63) Continuation of application No. PCT/CH03/00358, filed on Jun. 5, 2003.

(30) Foreign Application Priority Data
Jun. 12, 2002 (DE) 102 26 117.2

(51) Int. Cl. A61M 5/00

(52) U.S. Cl. 604/187

(57) ABSTRACT

The invention relates to coating or otherwise modifying or changing a surface characteristic, including a surface characteristic of a device for administering an injectable product, wherein the device includes a casing made of multiple casing parts, wherein at least one external surface of at least one of the casing parts is completely or partially provided with one or more surface-refining coatings. The coating may be applied to the casing part after or while the casing part is shaped, and the coating may comprise various materials which may be arranged alongside each other, on top of each other or may be mixed.

### CROSS-REFERENCE TO RELATED APPLICATIONS

This application is a continuation of International Patent Application No. PCT/CH03/00358, filed on Jun. 5, 2003, which claims priority to German Application No. DE 102 26 117.2, filed on Jun. 12, 2002, the contents of both applications are incorporated in their entirety by reference herein.

### FIELD

This invention relates to coatings and methods of and for treating, refining or modifying surfaces, and to devices and components of devices for administering injectable or infusable products. More particularly, it relates to devices for administering an injectable product, the devices comprising a casing wherein at least a portion of the casing has a refined surface.

### BACKGROUND

Devices such as injection or infusion devices are known for administering injectable substances. Such devices typically comprise a casing wherein the casing may further comprise multiple casing parts.

Individual casing parts of injection or infusion devices are generally manufactured using, for example, an injection molding process. Thus, the surfaces of the casing parts typically reflect the plastic raw material used for molding. The coloring, hardness, and roughness of the surfaces of the casing parts are, for example, dependent on the raw material. In order to adapt such characteristics to the requirements of the casing and/or injection or infusion device, various additives may be added to the raw material. Thus, for example, casing parts having different colors can be created by adding dyes. However, it has not hitherto been possible to create reflective or fluorescent surfaces of casing parts using such a method. Further, the addition of additives in the injection molding material can alter the characteristics of a casing part and its surface in a manner that may negatively influence other characteristics. The plastic used can, for example, become brittle and fragile, such that fractures arise in the surface.

Altering the characteristics of a surface by refining the surface has been done, for example, in the automobile industry. Coatings with selected characteristics may be used to generate protection against corrosion or to generate a particular design. In addition to conventional painting methods, more modern transfer methods are also being used to this end, such as, for example, diffusion, in-molding, and thermo-transfer methods.

An application for coating or modifying the surface of an injection pen is known from WO 98/55168. The casing of the injection pen is formed using a soft-touch material, i.e., a material that forms a softer surface than the material of the casing. The originally smooth surface of the casing is made easier to grip by the soft material, the soft material having a high friction coefficient and less force needs to be applied to perform the injection. To attach the coating, depressed areas into which the coating is fitted are provided on the casing. Thermoplastic elastomers, for example, block copolymers, are used as the material comprising soft-touch characteristics. The soft-touch material is attached to the casing by adhesion or mechanical fastening. This generally utilizes high temperatures which may negatively compromise the casing. The soft-touch material may separate from the raw material.

### SUMMARY

In one embodiment, the present invention relates to refining or modifying surface characteristics, including, refining or modifying at least a portion of the exterior surface of a device to provide a desired surface characteristic.

In one embodiment, the present invention relates to refining or modifying surface characteristics, including, refining or modifying at least a portion of the exterior surface of a device for administering an injectable product.

In one embodiment, the device may be, for example, an injection or infusion device. Such devices typically comprise a casing wherein the casing may further comprise multiple casing parts. In accordance with the invention, at least one casing part, or a portion of one casing part, of the casing comprises a refined surface. The refined surface may be an exterior surface of the casing part coated with a refining coating. The refined surface may improve individualism or distinctiveness of the device, improve the protection and handling of the device and/or increase the monitoring options when using the device. The device may be easily and flexibly adapted to different casing requirements and may have multiple configuration options.

A device for administering an injectable product in accordance with the present invention comprises a casing consisting of multiple casing parts, at least one of the casing parts being completely or partially provided with one or more surface-refining coatings. The casing part may comprise at least one surface which presents outwardly in the finished, assembled casing, and such outwardly facing surface may be provided with at least one surface-refining coating. The coating may applied to the casing part after or while the casing part is shaped, for example, by an injection molding process or in-molding process.

The coating comprises one or more materials wherein the materials may be applied alongside or on top of one another or may be mixed with one another within the coating.

The coating may comprise a paint. For example, the coating may comprise a paint, such as fluorescent, reflective, high gloss, a metallic dye, a primary dye, a primary dye with an additive of at least one other dye, or other paint. The coating may comprise a material that changes its color with a change in temperature. The coating may comprise a material having a lower thermal conductivity coefficient than the material of the casing. The coating may comprise a reflective material. The coating may comprise a softer material having a higher friction coefficient (soft-touch material) than the material of the casing. The coating may comprise a material having an odor additive. The coating may comprise a material having a tactile additive. The coating may comprise a material that forms a holographic representation. The coating may comprise a transparent material.

While multiple embodiments are disclosed, still other embodiments of the present teachings will become apparent to those skilled in the art from the following detailed description, which shows and described illustrative embodiments. As will be realized, the teachings are capable of modifications in various obvious aspects, all without departing from the spirit and scope of the present teachings. Accordingly, the drawings and detailed description are to be regarded as illustrative in nature and not restrictive.

### BRIEF DESCRIPTION OF THE DRAWINGS

FIG. 1 illustrates an injection device suitable for use in the present invention.

FIG. 2 illustrates the front section of FIG. 1 having a coating applied thereto in accordance with one embodiment of the present invention.

FIG. 3 illustrates another device suitable for use in the present invention, particularly another exemplary injection device.

FIG. 4 illustrates another device suitable for use in the present invention, particularly an exemplary infusion.

### DETAILED DESCRIPTION

The invention relates to changing, refining or modifying a surface characteristic, in one embodiment, as by applying a coating.

In one embodiment, the invention comprises refining at least a portion of the exterior surface of a device for administering an injectable product. The device may be, for example, an injection or infusion device, or a component thereof. Such device comprises a casing wherein the casing may further comprise multiple casing parts. In accordance with the invention, at least one casing part of the casing comprises a refined surface. The refined surface may be an exterior surface of the casing part coated with a refining coating. The refined surface may improve individualism or distinctiveness of the device, improve the protection and handling of the device and/or increase the monitoring options when using the device. The device may be easily and flexibly adapted to different casing requirements may have multiple configuration options.

A device for administering an injectable product in accordance with the present invention comprises a casing consisting of multiple casing parts, at least one of the casing parts being completely or partially provided with one or more surface-refining coatings. The casing part may comprise at least one surface which points outwardly in the finished, assembled casing and such outwardly facing surface may be provided with at least one surface-refining coating. The coating may applied to the casing part after or while the casing part is shaped, for example, by an injection molding process or in-molding process.

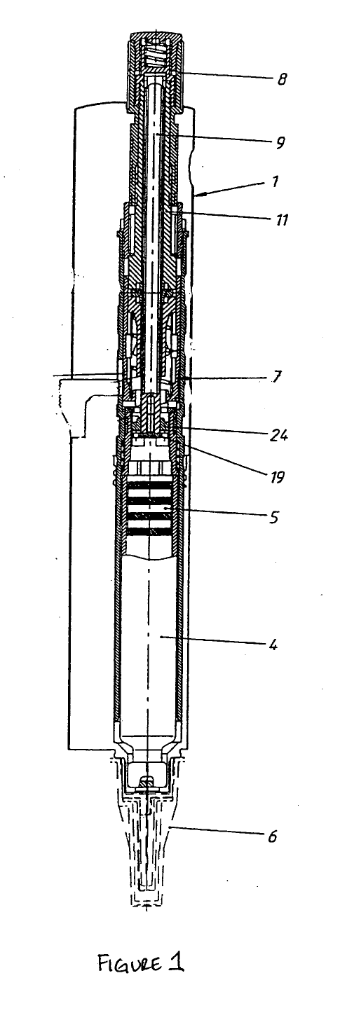

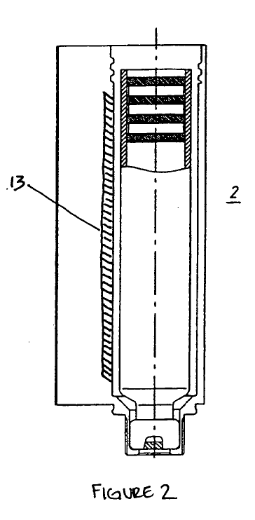

FIG. 1 illustrates an injection device suitable for use in the present invention. As shown, the injection device may comprise an injection pen. The injection device comprises multiple casing parts. As shown in FIG. 1, the injection device comprises a bushing-shaped main body 1 which can be divided into a rear (distal) section 3 containing the tubular actuating device or discharge mechanism 7 and a front (proximal) section 2 containing a replaceable ampoule 4 and a piston 5. A needle 6 with its distal end connected to the fluid to be administered can be screwed to the proximal end of the main body 1. The actuating device 7 comprises a control button 8, a threaded rod 9 with a flange 19, a guide member 24 and a driving member 11. As shown, each of rear section 3 and front section 2 include exterior surfaces to which a coating may be applied. FIG. 2 illustrates the front section 2 of FIG. 1 having a coating 13 applied thereto. U.S. Pat. No. 6,090,080, issued Jul. 18, 2000, to Jost et al., entitled “Injection Device for Injection of Liquid,” hereby incorporated by reference in its entirety, teaches an exemplary injection device for use in the present invention.

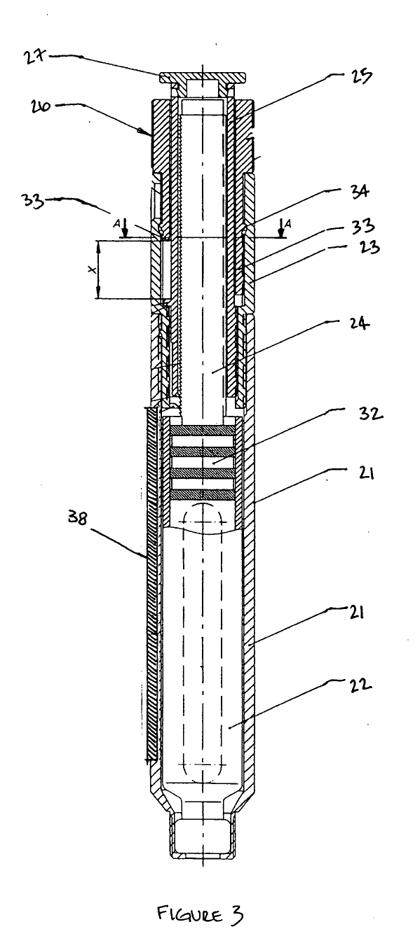

FIG. 3 illustrates another embodiment of an injection device suitable for use with the present invention. As shown, the injection device may comprise an injection pen. The injection device comprises multiple casing parts. The injection device shown in FIG. 3 consists of the ampoule holder 21 in which the ampoule 22 with the piston 32 and with the liquid substance to be administered are located. The sleeve-shaped mechanism holder 23 is mechanically coupled to the ampoule holder 21 by a thread or another suitable lock such as a bayonet lock. In the interior of the mechanism holder 23, the longitudinally shiftable shaft 24 is disposed acting on the piston 32 of the ampoule 22. The shaft 24 is surrounded by the also longitudinally shiftable advancing sleeve 25 mechanically coupled thereto, which is closed in its upper end area by the lid 27. For selecting a dose of the liquid substance to be injected, the dosing sleeve 26 with the stepped member 33 is provided which is arranged on the mechanism holder 23 in its upper end area. In this case, the dosing sleeve 26 with the stepped member 33 surrounds the advancing sleeve 25. The dose to be respectively adjusted and to be administered is selectable by a simple rotation of the dosing sleeve 26 with the stepped member 33. The dosing sleeve 26 is mechanically coupled to the mechanism holder 23 via the catch stop 34. FIG. 3 further illustrates a coating 38 applied to the surface of the ampoule holder 21. U.S. Pat. No. 6,277,101, issued Aug. 21, 2001, to Kirchhofer et al., entitled “Injection Device,” hereby incorporated by reference in its entirety, teaches another exemplary injection device for use with or in the present invention. Of course, an injection device for use in the present invention may vary from those shown.

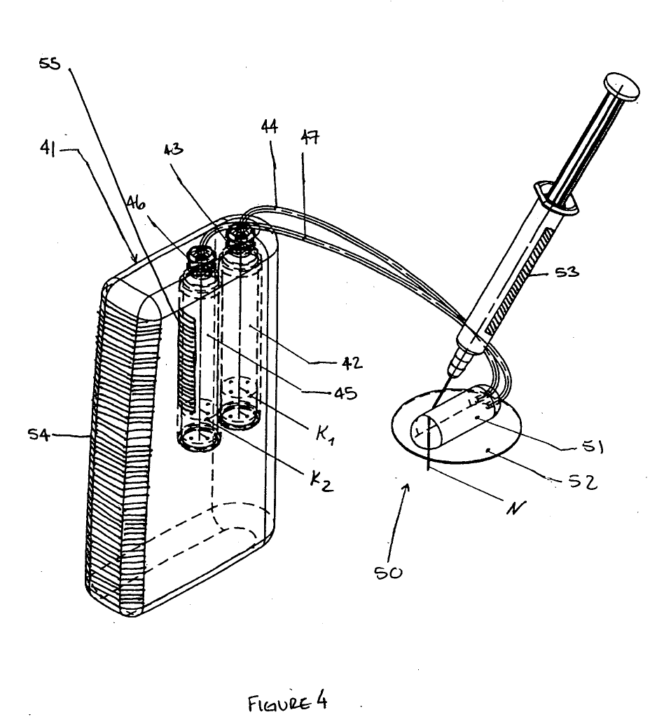

FIG. 4 illustrates an exemplary infusion device suitable for use in the present invention. As shown, the infusion device may comprise an infusion pump. The infusion device comprises multiple casing parts. Referring to FIG. 4, there is illustrated an embodiment of an infusor including two separate product fluid spaces. In a housing 41 of the infusor a first ampoule 42 and a second ampoule 45 are arranged adjacently parallel in a receiving chute. The housing 41 includes a coating 54 on at least one surface thereof. The receiving chute comprising straight guides for each of the ampoules 42 and 45 or is formed itself by two separate individual chutes. Each of the two ampoules 42 and 45 forms a separate product fluid space, i.e., a space closed off from the other space respectively, for the product fluid accommodated therein. Each of the two ampoules 42 and 45 may be formed by a conventional syringe ampoule. As shown, one of the ampoules 45 includes a coating 55 on a surface thereof. Accommodated in ampoule 42 is a piston K1 and in ampoule 45 a piston K2 which are advanced to the corresponding ampoule outlet 43 and 46 respectively by means of a delivery means, preferably comprising motor-powered spindle drives to dispense the corresponding product fluid through the corresponding outlet 43 and 46 in the course of the advance movement. The pistons K1 and K2 form the components of the delivery means acting directly on the product fluid. Connected by conventional means to each of the fluid outlets 43 and 46 is a fluid conduit 44 and 47 respectively. The fluid conduits 44 and 47 are simple catheters and are formed conventionally by a separate flexible tube with a Luer-lock tip to the ampoule. Each of the separate fluid conduits 44 and 47 ports separately into a common catheter head 20.

The catheter head 50 comprises a two-part catheter head housing 51 including a flat underside or contact plate 52. Protruding from the underside at the proximal end of the catheter head housing 51 is a piercing needle N. The needle end may be generally perpendicular with respect to the underside. Within the catheter head 50, the fluid conduits run separate up to the catheter head and, formed by the single catheters 44 and 47, are merged prior to the piercing needle N. The underside of the catheter head 50 is prepared for attaching the catheter head 50 to the skin of the user. A syringe may be used with the device. The syringe includes a coating 53 on a surface thereof.

As shown, the infusion device includes a plurality of casing parts and each of these casing parts may include an exterior surface. For example, housing 41, ampoule 46, ampoule 43 each comprise exterior surfaces, and any of these or other surfaces can be treated or refined in accordance with the present invention. Interior surfaces may be similarly treated or refined. U.S. Pat. No. 6,620,138, issued Sep. 16, 2003, to Marrgi et al., entitled “Infusor, Catheter Means and Catheter Head,” hereby incorporated by reference in its entirety, teaches an exmplary infusion device for use in the present invention. Of course, an infusion device for use with or in the present invention may vary from that shown.

As discussed above, FIGS. 1 through 4 illustrate ampoules suitable for use with injection or infusion devices. Any such ampoule may comprise an exterior surface which may be coated to provide a refined surface, in accordance with the present invention. Further, any such ampoule may contain an injectable product and may be configured for insertion into an injection or infusion device.

In one embodiment, a refined surface in accordance with the present invention comprises a surface having a coating applied thereto. In one embodiment, the surface is part of a casing part of an injection or infusion device having a coating applied thereto. The coating may comprise one or more materials wherein the materials may be applied alongside or on top of one another, or may be mixed with one another within the coating. Suitable materials for the coating include paints, such as fluorescent, reflective, high gloss, a metallic dye, a primary dye, a primary dye with an additive of at least one other dye, or other paint, materials that change color with a change in temperature, materials having a lower thermal conductivity coefficient than the material of the casing, reflective materials, materials having a higher friction coefficient (soft-touch material) than the material of the casing, materials having an odor additive, materials having a tactile additive, materials that form holographic representations, transparent materials, or other materials.

Thus, in one embodiment of the invention, the coating comprises a paint. Injection or infusion devices are frequently identical or very similar, having no distinguishing characteristics from another of the same device. Refining the surface of casing parts with a paint coating enables a number of different forms or appearances to be achieved. This individualizes or personalizes a device, for example, an injection pen, such that a user can identify their injection pen.

Injection molding is typically used to manufacture an injection or infusion device. Certain limitations arise with injection molding. A paint coating enables novel configurations for the casing of the injection or infusion device not generally available using injection molding. For example, using injection molding, fluorescent or reflective casing surfaces are not achievable. By applying a paint comprising a fluorescent, reflective, high gloss or metallic dye, new surface configurations are achievable. Thus, the paint may comprise fluorescent, reflective, high gloss, a metallic dye, a primary dye, a primary dye with an additive of at least one other dye, or other paint.

In another embodiment, the coating comprises a material that changes its color with a change in temperature. Thus, for example, heat-sensitive plastics, heat sensitive liquid crystals, or heat-sensitive paints may be used as coatings. The change in color of the material may remain permanent as a new tint of the surface, even if the temperature returns to the original temperature again. Alternately, the color of the material may return to the original color when the temperature returns to the original temperature. Using such a temperature-sensitive coating on the casing of the injection or infusion device, the temperatures to which the device and therefore also the injectable product has been exposed can be monitored. Medical, pharmaceutical or cosmetic products can be highly temperature-sensitive and it is possible that, once excessively heated or cooled, they are no longer suitable for administering. Such excessive heating or cooling of the product may be indicated by the change in color of the casing of the injection or infusion device.

Thus, the present invention provides an injection or infusion device including at least one casing part having a coating comprising a material that changes its color with a change in temperature. For example, the casing part may be the casing part containing the injectable product. In one embodiment, the material is configured such that the change of color of the material due to temperature remains permanent even upon return of the temperature to the original temperature. Thus, the coating indicates to a user that the casing part has been exposed to a temperature sufficient to change the color of the coating.

In another embodiment, the coating comprises a material having a lower thermal conductivity coefficient than the material of the casing. A coating comprising a material having a lower thermal conductivity coefficient than the material of the casing thermally insulates the casing part thus coated. The thickness of the thermally insulating coating may be adapted to specific insulating requirements. Rubber or latex materials can, for example, be used to this end. A coating consisting of fibrous material or textiles can, for example, also be used for temperature insulating. Thus, the invention provides an injection device wherein the region of the casing surrounding the product to be injected is coated with a thermally insulating material, in order to protect the product against heating or cooling influences.

As stated above, the present invention further includes providing more than one coating on an injection or infusion device. Thus, in one embodiment, a region of the casing surrounding the product to be injected is coated with a thermally insulating material while another part of the casing of the injection or infusion device is provided with a coating consisting of a material comprising a temperature-dependent or heat-sensitive dye. This embodiment may be useful, for example, with an injectable product that is sensitive to cold or heat. The thermally insulating material protects the injectable product against heating or cooling influences while the material comprising a temperature-dependant indicates whether the injectable product has been excessively heated or cooled despite the insulating layer and is therefore possibly no longer suitable for injecting.

In another embodiment, the coating comprises a reflective material. A reflective material as a surface-refining coating of a casing part in accordance with the invention not only achieves new optical effects on the surface of the casing, but also serves to thermally insulate the injectable product within the casing. Providing a coating comprising a reflective material thermally insulating the casing part thus coated. The invention thus provides a casing part for containing an injectable product, the casing part being coated with a reflective material to thermally insulate the injectable product. Any suitable reflective material—for example, reflective films, fabrics, paints, tapes, or other—may be used.

In another embodiment, the coating comprises a material having a higher friction coefficient (soft-touch material) than the material of the casing. Synthetic elastomers may be considered as the soft-touch material for increased grip. Such coating may be used to increase the grip of the injection device, such that a lower application of force is required when performing an injection. Any soft-touch material may be used. For example, a rubber or plastic material may be used so long as the material has a higher friction coefficient than the material of the casing.

If the injection device has smooth surfaces, it is possible that, when pressing the injection device onto the location at which the product is to be injected, the device can slip from the user's grip. To avoid losing grip of the device, the user may have to apply an increased pressure and exercise increased caution. Coating a gripping surface of the device with a soft-touch material minimizes the possibility of the device slipping from the user's grip and makes using the injection or infusion device simpler and safer. Thus, the invention provides an injection or infusion device wherein the casing part intended for gripping by the user is coated with a soft-touch material.

In another embodiment of the invention, at least one impact edge of a casing part is provided with a soft-touch coating. When assembling the individual casing parts to form the overall casing of the injection or infusion device, this coated impact edge abuts the adjacent casing part to form a seal for the overall casing. The soft-touch material thus forms a tight seal with the adjacent edge. It is also possible to coat all the impact edges of the casing parts with the soft-touch material, such that the soft-touch surfaces abut one another and form a tight seal at the edges.

In another embodiment, the coating comprises a material having an odor additive. Various olfactives may be considered as odor additives, enabling one injection or infusion device to be distinguished from another by its characteristic odor. This may be advantageous for users with weak sight. Thus, the invention provides an injection or infusion device having at least one casing portion coated with a material having an odor additive in order to provide olfactory identification to the device. Thus, for example, a fragrance may be added to a coating.

In another embodiment, the coating comprises a material having a tactile additive. Thus, for example, the material may be provided with granular particles. Adding granular particles in the coating material increases the roughness of the surface. If such a material is arranged at particular selected points on the surface of a casing, the injection or infusion device can be identified using the sense of touch. Thus, the invention provides an injection or infusion device having at least one casing portion coated with a material having a tactile additive in order to provide tactile identification to the device. Further, a casing part such as ampoule may be coated with a material having a tactile additive wherein several ampoules having different injectable products therein are differently coated.

In another embodiment, the coating comprises a material that forms a holographic representation. Such a hologram on the casing may be used to increase security against the injection or infusion device being forged. Further, a selection of holograms may be provided, thus enabling increased personalization of the device. The invention thus provides an injection or infusion device comprising at least one casing part being coated with a material forming a holographic representation to increase security of the device or to increase personalization of the device.

The coating may comprise a transparent material. The transparent coating may aid in visually monitoring the product.

By refining the surface of an injection or infusion device using one or more of materials, such as those described above, the device may be adapted to particular preferences provided, for example, by the user or by the peculiarities of a product to be injected. These preferences can be met by selecting a material that has a suitable surface refining or modifying functionality. Due to the variety of the individual surface refining materials, a coating may also combine various functions on the same casing, by coating different casing parts or different regions on a single casing part with various materials.

In accordance with the invention, the coating may be applied to a casing part, already formed, for example, by injection molding. The characteristics of the raw material of the casing part are not negatively influenced and the quality of the casing part formed is not reduced through the addition of a coating refining the surface. Using a paint as the starting material of refining the surface may aid in ensuring that the casing surface of the injection molded part is suitably connected to the coating material.

In applying the coating to the casing part, a paint may be applied as a base material of the coating. The paint may be applied to the surface of a casing part, for example, by spray-painting or immersion-painting. Paints further represent a surface that is easy to clean and that is not typically corroded by chemicals, for example, cleaning agents.

Other possible base materials for the surface-refining coating include conventional plastics, polymers, or copolymers, etc. If plastic films are used as the surface-refining coating, these may be applied to the casing part by being laminated or calendared.

The surface-refining coating may comprise of a prefabricated film applied, for example, by adhesion, printing or in-molding methods. Methods such as vacuum evaporation, adhesion methods, transfer methods, thermo-transfer methods, spray-painting methods, printing methods, immersion painting methods, airbrush methods, in-molding methods or other suitable methods may be used to apply the coating to casing parts.

In another embodiment of the invention, the casing part coated with the refined surface is arranged, as an additional part, on a functional casing. This may be achieved, for example, by clicking or pushing on an additional part adapted to the form of the functional casing. Further, particular regions on an already functional casing may be provided for receiving such an additional part. Thus, for example, recesses may be provided on the functional casing for receiving the additional part. Similarly, protrusions may be provided on the functional casing that the additional part abuts. This enables injection or infusion devices which can be manufactured uniformly to be distinguished from each other by differently coated additional parts, or already existing devices to be retro-fitted with such an additional part. Such an additional part may be easily exchanged, enabling the same device to easily be given a new appearance.

By refining the surface of an injection or infusion device using one or more of the materials of the present invention, the device may be protected from external influences, use of the injection device may be simplified, and the acceptability of such an injection or infusion device to a user may facilitated by personalizing it for the user.

Although the present invention has been described in reference to preferred embodiments, persons skilled in the art will recognize that changes may be made in form and detail without departing from the spirit and scope of the invention.

1. A device for administering an injectable product, comprising:
a casing comprising multiple casing parts, at least one of the casing parts including a surface;
a coating, wherein the coating is applied to the surface of the at least one casing part, after or while the casing part is shaped; wherein
the coating changes a surface characteristic of the surface to which it is applied.

2. The device of claim 1, wherein said changes in surface characteristics comprise at least one of improved individualism or distinctiveness of the device, improved protection and handling of the device, and increased monitoring options when using the device.

3. The device of claim 1, wherein the coating comprises a paint.

4. The device of claim 2, wherein the paint is a fluorescent paint.

5. The device of claim 2, wherein the paint is a metallic dye.

6. The device of claim 2, wherein the coating comprises a material that changes color with a change in temperature.

7. The device of claim 6, wherein the change in color is permanent.

8. The device of claim 2, wherein the coating comprises a material having a higher friction coefficient than a base material of the casing part to which the coating is applied.

9. The device of claim 8, wherein the coating is applied over at least one impact edge of at least one casing part.

10. The device of claim 2, wherein the coating comprises a thermally insulating material.

11. The device of claim 10, wherein the coating comprises a reflective material.

12. The device of claim 10, wherein the coating comprises a fibrous material.

13. The device of claim 2, wherein the coating comprises a material having a lower thermal conductivity coefficient than a material of the casing part to which it is applied.

14. The device of claim 2, wherein the coating comprises a material provided with an odor additive.

15. The device of claim 2, wherein the coating comprises a material provided with a tactile additive.

16. The device of claim 1, wherein the coating comprises a material that forms a holographic representation.

17. The device of claim 1, wherein the surface is completely coated.

18. The device of claim 1, wherein the surface is partially coated.

19. The device of claim 1, wherein a plurality of casing parts are provided with coatings on exterior surfaces thereof and wherein the coatings on the different casing parts are different.

20. The device of claim 1, wherein a first casing part is provided with a coating comprising a thermally insulating material and a second casing part is provided with a coating that changes color with temperature.

21. The device of claim 1, wherein the coating comprises a prefabricated film.

22. The device of claim 1, wherein the coated casing part is arranged, as an additional part, on a functional casing.

23. The device of claim 1, wherein the coating is applied to the casing part by spray-painting, immersion-painting, adhesion, printing, vacuum evaporation, thermo-transfer methods, in-molding methods or airbrush methods.

24. The device of claim 1, wherein the device is an injection apparatus.

25. The device of claim 1, wherein the device is an infusion apparatus.
